(19) United States
(12) Patent Application Publication
(10) Pub. No.: US 2011/0218428 A1
(43) Pub. Date: Sep. 8, 2011

(54) System and Method for Three Dimensional Medical Imaging with Structured Light

(75) Inventors: Robert Joe Westmoreland, Heath, TX (US); Michael Spencer Troy, San Antonio, TX (US)

(73) Assignee: MEDICAL SCAN TECHNOLOGIES, INC., Heath, TX (US)

(21) Appl. No.: 13/040,952

(22) Filed: Mar. 4, 2011

Related U.S. Application Data
(60) Provisional application No. 61/310,621, filed on Mar. 4, 2010.

(51) Int. Cl. A61B 6/00

(52) U.S. Cl. 600/425

(57) ABSTRACT

An SLI medical image sensor system captures one or more images of a skin lesion and generates a 3D surface map of the skin lesion using SLI techniques. A feature detection module processes the 3D surface map to detect certain characteristics of the skin lesion. Feature data of the skin lesion is generated such as size, shape and texture. A feature analysis module processes the feature data of the skin lesion. The feature analysis module compares the skin lesion to prior images and feature data for the skin lesion. The feature analysis module categorizes the skin lesion based on templates and correlations of types of features.

### CROSS-REFERENCE TO RELATED PATENTS

The present U.S. Utility patent application claims priority pursuant to 35 U.S.C. §119(e) to U.S. Provisional Application Ser. No. 61/310,621, entitled, “System and Method for Three Dimensional Medical Imaging with Structured Light,” filed Mar. 4, 2010, which is incorporated by reference herein and made part of the present U.S. Utility patent application for all purposes.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not applicable.

### BACKGROUND OF THE INVENTION

1. Technical Field of the Invention

This invention relates to three dimensional (3D) medical imaging and in particular to systems and methods for medical imaging of melanoma using structured light illumination.

2. Description of Related Art

Structured light illumination (SLI) techniques are a relatively low cost method for generating 3D images in biometrics, e.g. fingerprint and facial recognition. For example, one method is described in PCT Application No. WO2007/050776 entitled, “System and Method for 3D Imaging using Structured Light Illumination,” which is incorporated by reference herein. See also, U.S. Pat. No. 7,440,590 entitled, “System and Technique for Retrieving Depth Information about a Surface by Projecting a Composite Image of Modulated Light Patterns,” which is incorporated by reference herein. See also, US Published Application No. 20090103777 entitled, “Lock and Hold Structured Light Illumination,” which is also incorporated by reference herein. SLI imaging techniques have proven a cost effective solution in biometrics.

As disclosed herein, it is desirable to apply SLI imaging techniques in other fields to provide relatively low cost and fast 3D imaging.

### BRIEF DESCRIPTION OF THE SEVERAL VIEWS OF THE DRAWINGS

FIG. 1 illustrates a schematic block diagram of an embodiment of a Structured Light Illumination (SLI) medical image system;

FIG. 2 illustrates a schematic block diagram of an embodiment of a Structured Light Illumination (SLI) medical image sensor;

FIG. 3 illustrates a schematic block diagram of another embodiment of a Structured Light Illumination (SLI) medical image sensor;

FIG. 4 illustrates a schematic block diagram of an embodiment of a projection system in a SLI medical image sensor;

FIG. 5 illustrates a schematic block diagram of an embodiment of a medical image camera system in a SLI medical image sensor;

FIG. 6 illustrates a logical flow diagram of an embodiment of a method for capturing medical images using SLI techniques;

FIG. 7 illustrates a logical flow diagram of an embodiment of a method for generating a 3D surface map from SLI medical image data;

FIG. 8 illustrates an example of a 3D surface map generated from SLI image data;

FIG. 9 illustrates a logic flow diagram of an embodiment for processing a 3D surface map to generate anatomical feature data;

FIG. 10 illustrates a logic flow diagram of an embodiment for using SLI techniques in dermatology;

FIG. 11 illustrates a logic flow diagram of an embodiment of a method for processing skin feature data;

FIG. 12 illustrates a logic flow diagram of an embodiment of another method for processing skin feature data;

FIG. 13 illustrates a schematic block diagram of an embodiment of a skin feature detection module;

FIG. 14 illustrates a schematic block diagram of an embodiment of a skin lesion analysis module; and

FIG. 15 illustrates a logic flow diagram of an embodiment of another method for processing skin feature data.

### DETAILED DESCRIPTION OF THE INVENTION

A need exists to provide a method and system for use of Structured Light Illumination (SLI) techniques in medical imaging systems of anatomical features, and in particular in imaging of melanoma and other skin lesions. SLI medical imaging systems described herein provide for cost effective and fast imaging, comparison, classification and analysis of anatomical features.

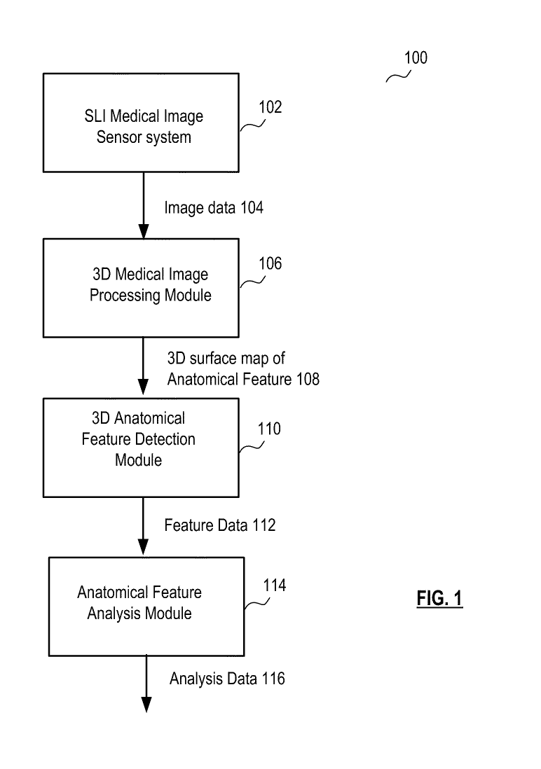

FIG. 1 illustrates a schematic block diagram of an embodiment of an SLI medical imaging system 100. An SLI medical image sensor system 102 captures one or more two dimensional images of an anatomical feature and generates 3D medical image data 104 of the anatomical feature. The anatomical feature is any feature of or relating to the human body or animal body, such as skin, body parts and eyes and in an embodiment, skin lesions such as melanoma.

The 3D medical image processing module 106 processes the 3D medical image data 104 and generates a 3D surface map of the anatomical feature 108. A feature detection module 110 processes the 3D surface map 108 to detect certain characteristics of the anatomical feature. Feature data 112 of the anatomical feature is generated such as size, shape and texture. An anatomical feature analysis module 114 processes the feature data 112. In an embodiment, the anatomical feature analysis module 114 compares the anatomical feature to prior images and feature data for the anatomical feature. The feature analysis module 114 categorizes the anatomical feature based on templates and correlations of types of features.

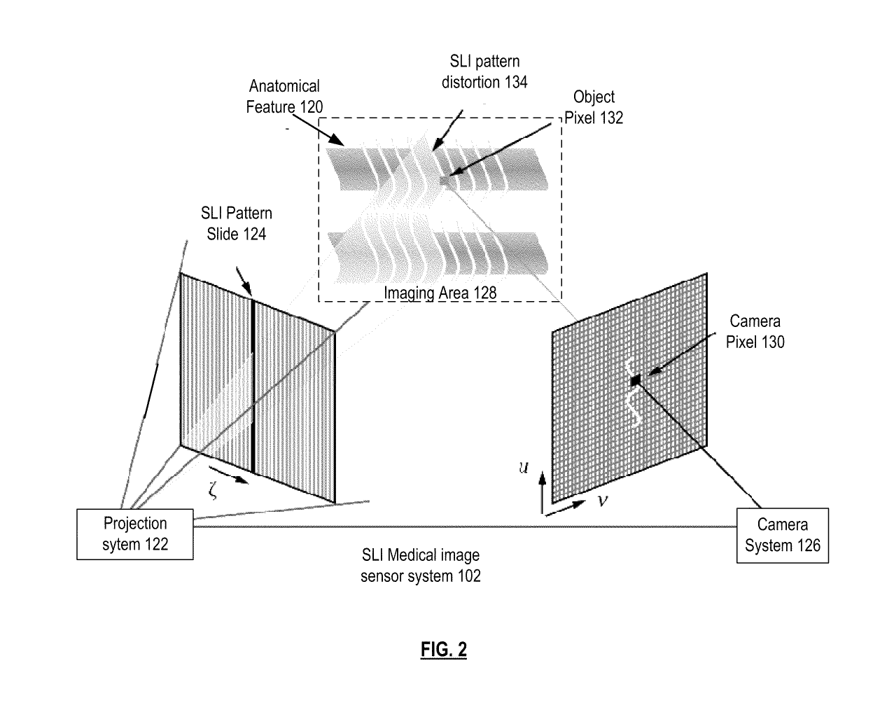

FIG. 2 illustrates a schematic block diagram of an embodiment of the SLI medical image sensor system 102. In an embodiment, the medical image sensor system 102 includes Structured Light Illumination (SLI) technology. The SLI medical image sensor system 102 includes an SLI pattern projection system 122 and camera system 126. The SLI pattern projection system 126 includes a DLP projector, LCD projector, LEDs, or other type of projector or laser. The camera system 126 includes one or more digital cameras or other type of image sensors operable to capture digital images. In an embodiment, the camera system 126 is a microscopic camera able to capture images on a micron scale. Though illustrated in one position, multiple cameras may be positioned at different angles to the imaging area and projection system 122.

In operation, the one or more cameras in camera system 126 are focused onto the imaging area 128. The projection system 122 projects focused light through an SLI pattern slide 124 onto an anatomical feature 120 in imaging area 128. The SLI pattern is distorted by the surface variations of the anatomical feature 120 as seen with SLI pattern distortion 134. While the SLI pattern is projected onto the anatomical feature 120, the camera system 126 captures at least one image of the anatomical feature 120 with the SLI pattern distortion 134. The camera system 126 generates a frame composed of a matrix of camera pixels 130 wherein each camera pixel 130 captures image data for a corresponding object point 132 on the anatomical feature 120. The camera system 126 captures one or more images of the anatomical feature 110 with the distortions in the structured light pattern. Additional SLI slide patterns may be projected onto the anatomical feature 120 while additional images are captured. The one or more images are then stored in a medical image data file for processing.

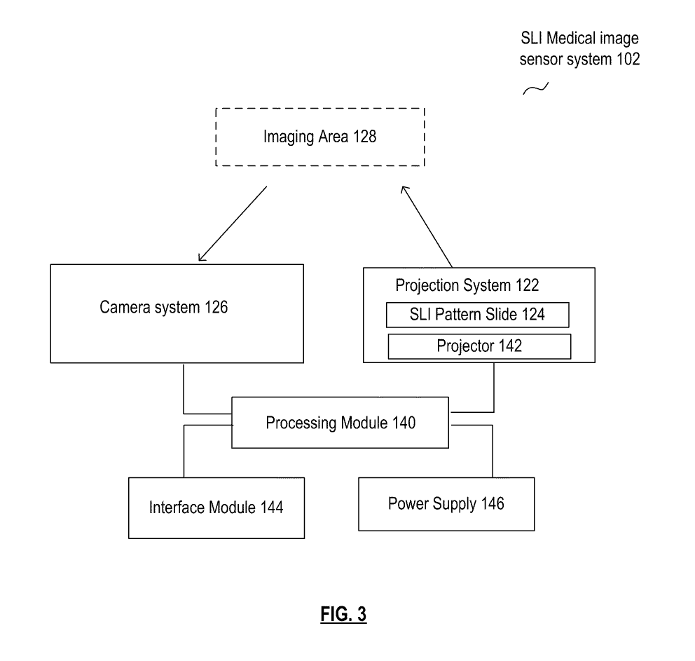

FIG. 3 illustrates another schematic block diagram of an embodiment of a Structured Light Illumination (SLI) medical image sensor system 102. The medical image sensor system 102 includes a camera system 126, projection system 122, processing module 140, interface module 144 and power supply 146. The power circuit and projection system are designed in an embodiment to provide illumination under a variety of ambient lighting conditions. The camera system 126 includes one or more image sensors operable to capture images of an anatomical feature. Projection system includes one or more digital light projectors (DLP) projectors 142 and one or more SLI pattern slides 124. Alternatively, laser lights may be programmed to project a certain SLI pattern onto the anatomical feature. The power supply 146 is coupled to the camera system 126, projection system 122 and processing module 140. The interface module 144 provides a display and user interface, such as keyboard or mouse, for monitoring and control of the SLI medical image sensor system 102 by an operator. The interface module 144 may include other hardware devices or software needed to operate the image sensor system 102 and provide communication between the components of the image sensor system 102.

The processing module 140 includes one or more processing devices, such as a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. The processing module includes a memory that is an internal memory or an external memory. The memory of the processing module 106 may each be a single memory device or a plurality of memory devices. Such a memory device may be a read-only memory, random access memory, volatile memory, non-volatile memory, static memory, dynamic memory, flash memory, cache memory, and/or any device that stores digital information. When processing module may implements one or more of its functions via a state machine, analog circuitry, digital circuitry, and/or logic circuitry, the memory storing the corresponding operational instructions may be embedded within, or external to, the circuitry comprising the state machine, analog circuitry, digital circuitry, and/or logic circuitry. Processing module may execute hard coded and/or operational instructions stored by the internal memory and/or external memory to perform the steps and/or functions illustrated in FIGS. 1 through 15 described herein. The processing module and the interface module may be integrated into one or more devices or may be separate devices.

In operation, an anatomical feature is imaged in the imaging area 128 by the camera system 126 while one or more SLI patterns are projected onto the anatomical feature by the projection system 122. The processing module 140 (or camera system 126) includes a timing circuit to ensure proper timing of capturing of images by the camera system 126 and projection of the SLI pattern into the imaging area by the projection system 122. The anatomical feature may move through the imaging area 128 or camera system 126 may be moved to capture the desired anatomical features.

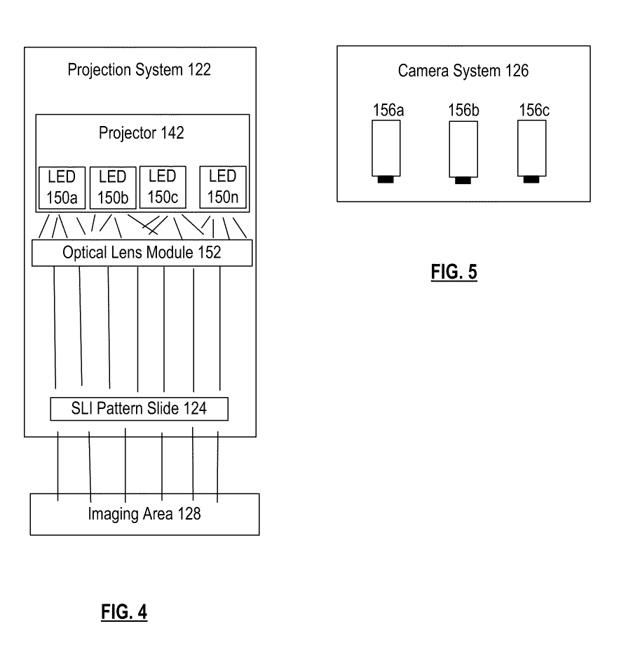

FIG. 4 illustrates a schematic block diagram of an embodiment of a projection system 122 for use in the image sensor system 102. In this embodiment, the projector 142 includes an array of high intensity light emitting diodes (LED) 150a-n. The LEDs 150a-n are triggered for a pulse duration sufficient to provide ample exposure at the highest frame rate of the camera system 126, while minimizing the duration to avoid motion blur of the anatomical feature during the exposure. The use of an array of LEDs 150 rather than a DLP projector in this embodiment reduces hardware cost and size of the SLI system 100. Using a high intensity LED array as a flash unit also allows for increased image signal to noise ratio (SNR) and shorter exposure times. The LEDs 150 are selected to match the spectral characteristics of the camera sensor system 126. In another embodiment, the projection system 122 may include a DLP projector or other type of projector.

The projection system 126 includes optical lens module 142. The optical lens module 142 projects the light from the LEDs through the SLI pattern slide and focuses the SLI pattern into the imaging area. The optical lens module 142 helps to evenly spread the light that is emitted by the high-power LEDs and then to focus the light on the imaging area. In an embodiment, the optical lens module 142 focuses light only in the axis perpendicular to the LED array, achieving further efficiency in light output by only projecting light in an aspect ratio that matches that of the pattern slide. For example, the optical lens module may include one or more cylindrical lenses.

FIG. 5 illustrates a schematic block diagram of an embodiment of camera system 126 for use in the image sensor system 102. The camera system 126 includes one or more image sensors 156. In an embodiment, the image sensors are CCD (Charge coupled device) camera modules, CMOS (Complementary metal-oxide-semiconductor) camera modules, or other type of image sensor modules. In an embodiment, the image sensors 156 include a digital camera for microscopy with accompanying microscope that may capture images on a micron scale. The image sensors 156 include a high speed data interface, such as USB interface, and include a trigger input for synchronization with the projection system 122. Each image sensor 156 may include its own lens or a single lens may be used for focusing each of the image sensors 156.

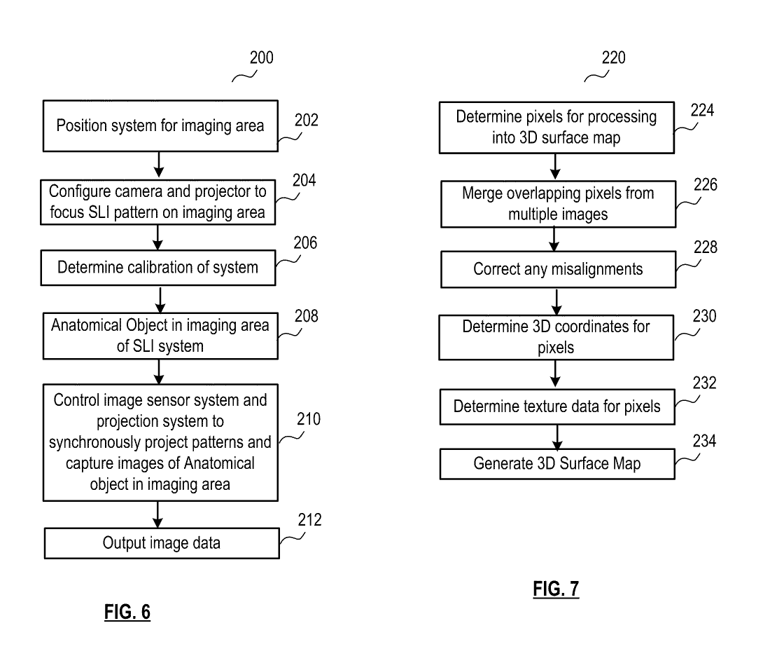

FIG. 6 illustrates a logical flow diagram of an embodiment of a method 200 for capturing medical images using SLI techniques. In operation, the medical image sensor system 102 is positioned for the desired imaging area in step 202. In step 204, the camera system 126 and projection system 122 are configured to focus onto the imaging area 128. The projection system 122 projects focused light through an SLI pattern slide on the imaging area 128 while the camera system 126 captures images. System calibrations are determined in step 206. In step 208, the desired target area of the anatomical feature is positioned in the imaging area. In step 210, while the SLI pattern is projected onto the anatomical feature by the projection system 122, the camera system 126 captures one or more images of the anatomical feature with SLI pattern distortion. The camera system 126 captures one or more images of the anatomical feature with the distortions in the structured light pattern. Additional SLI slide patterns may be projected onto the anatomical feature while additional images are captured. Image data for the one or more captured images is then stored in a medical image data file for processing in step 212.

The 3D medical image processing module 106 shown in FIG. 1 processes the image data for the one or more captured images. FIG. 7 illustrates a logical flow diagram of an embodiment of a method 220 for generating a 3D surface map from the image data. In step 224, the image data is processed by determining pixels in the captures images for processing. For example, the images are segmented to eliminate unwanted pixels or points or data. The segmentation technique includes background-foreground modeling to eliminate background image data from a region of interest. The background-foreground modeling is performed as part of a training stage by collecting a number of background images and computing the average background model image. The foreground image information is extracted by labeling any image pixel that does not lie within a specified tolerance of the average background model image. The segmented image data is used to determine the surface map. In step 226, if multiple images were captured of a targeted area, pixels representing the same object point from overlapping images are aligned. This image data extracted from the segmented foreground and aligned across the multiple images is used determine the 3D points on a surface map. For example, a skin lesion is extracted from background points or separated from other points of the skin. If the skin lesion is in multiple images, common points from the images are aligned to obtain all image data for the object points of the skin lesion. In step 228, any misalignments are corrected.

The distortions in the structured light pattern in the captured images are analyzed and calculations performed to determine a spatial measurement of various object points of the anatomical feature in step 230. This processing of the images uses well-known techniques in the industry, such as standard range-finding or triangulation methods. The triangulation angle between the camera and projected pattern causes a distortion directly related to the depth of the surface. Once these range finding techniques are used to determine the position of a plurality of points on the surface of the anatomical feature, then a 3D data representation of the anatomical feature can be created. An example of such calculations is described in U.S. Pat. No. 7,440,590, entitled, “System and Technique for Retrieving Depth Information about a Surface by Projecting a Composite Image of Modulated Light Patterns,” by Laurence G. Hassebrook, Daniel L. Lau, and Chun Guan filed on May 21, 2003, which is incorporated by reference here. The 3D coordinates for a plurality of object points is determined Collectively, the plurality of points is called a 3D surface map. Each point in the 3D surface map is represented by 3D coordinates, such as Cartesian (x, y, z) coordinates, spherical (r, θ, Φ) coordinates or cylindrical (y, r, θ) coordinates.

In addition, each point includes texture data. Texture data includes color values, such as Red, Green and Blue values. Texture data also includes grey values or brightness values as well. Texture data for the points in the 3D surface map are determined in step 232 and in step 234, the 3D surface map of the anatomical feature is generated.

Various SLI techniques and SLI patterns may be implemented in the SLI medical image sensor system 102 described herein. For example, see PCT Application No. WO2007/050776, entitled System and Method for 3D Imaging using Structured Light Illumination, which is incorporated by reference herein. See also, US Published Application No. 20090103777, entitled Lock and Hold Structured Light Illumination, which is also incorporated by reference herein. See also, PCT application Ser. No. 09/43056, entitled “System and Method for Structured Light Illumination with Frame Subwindows,” filed on May 6, 2009, which is incorporated by reference herein.

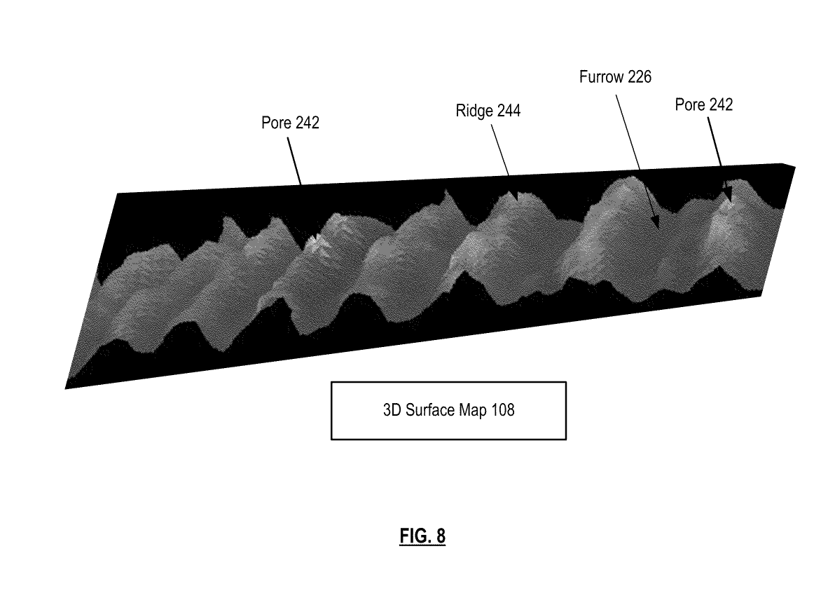

FIG. 8 illustrates an example of a 3D surface map 108 generated from SLI image data. In this example, the 3D surface map 108 includes pores 240, ridges 244 and furrows 226 from skin of a fingertip. Since the 3D surface map 108 includes 3D coordinates of each of the points in the surface map, the size and shape of various features can be measured, such as the size and shape of a pore or mole on the skin. Texture data, such as color and intensity (e.g., brightness), of a feature can also be determined from the 3D surface map 108.

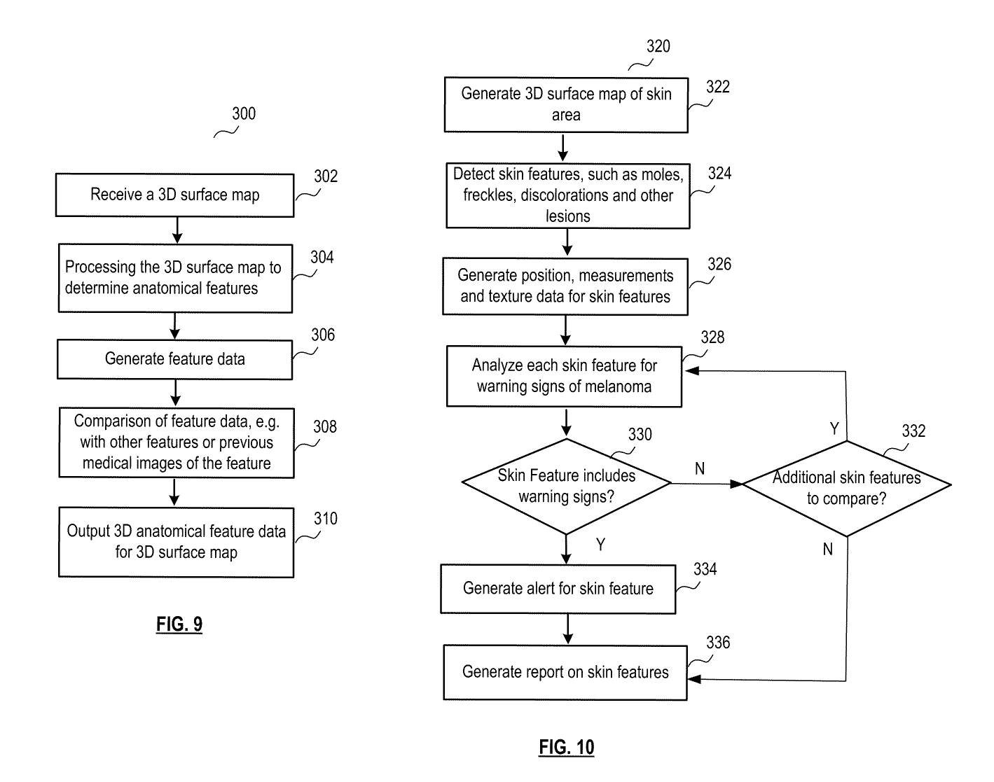

FIG. 9 illustrates a logic flow diagram of an embodiment of a method 300 for processing the 3D surface map to generate anatomical feature data. Once a 3D surface map is generated and/or received in step 302, anatomical features present in the 3D surface map can be determined in step 304. Depending on the type of feature, e.g. type of lesion such as scar, freckle, bump, etc., the 3D surface map is compared to various feature templates to determine the type of feature. Various feature data can then be determined for the identified type of lesion in step 306. For example, the feature data may include size, density, volume, shape, color, etc. In step 308, the detected feature data is compared with feature data from previous SLI scan images to determine changes over time. Changes, such as in size, density, shape and color, can be objectively measured. In an embodiment, the feature data is compared with other feature templates to determine warning signs or abnormalities in the feature. The feature may be compared to feature templates of average features, feature templates of diseased features or to a correlation of feature data from the same person. In step 310, the feature data for the detected features in the 3D surface map and any results of comparisons are generated. The results of the comparison can be provided to a medical expert for interpretation and review.

FIG. 10 illustrates a logic flow diagram of an embodiment of a method 320 for using SLI techniques in dermatology to detect skin lesions. The SLI system described herein provides a lower cost system to assist in early detection and monitoring of skin lesions for signs of melanoma or other skin disease. Due to high costs, current imaging systems are not affordable for the average doctor's office. In addition, current imaging costs are too expensive for annual visits or regular check-ups. Due to its lower costs, the SLI medical imaging system described herein is affordable and cost effective solution for imaging at annual visits and check-ups in a doctor's office.

The SLI medical image sensor system 102 images an area of skin, and the image processing module 106 generates a 3D surface map of the skin area in step 322. The feature detection module 110 then detects skin features, such as moles, freckles, discolorations and other lesions, from the 3D surface map in step 324 and extracts the points for selected skin features for further analysis. Various feature data for a selected skin lesion is determined from the 3D surface map. For example, position, size measurements, density measurements, shape measurements and texture data for one or more of the selected skin features is determined in step 326.

In an embodiment, the skin feature analysis module 114 compares each skin feature for warning signs of melanoma in step 328, such as discolorations, irregular border, asymmetrical shape and large size. When such a characteristic is detected in a skin feature in step 330, an alert is provided with the feature data in step 334. A physician can review the 3D image, 2D image and/or feature data and determine a proper course of action. In step 322, the system determines whether additional skin features are to be analyzed. If yes, the process continues at step 328. If not, then a report on the skin features and feature data is generated in step 336.

In an embodiment, the SLI medical imaging system is used to image skin areas for melanoma screening. Due to its low cost, medical imaging for melanoma screening at each check-up or annual visit is now affordable. Currently, subjective review of skin areas is made by a physician without imaging. There is no record of prior images so growth cannot be detected. It is difficult to screen each skin feature and identify discolorations and other characteristics over a large skin area by a physician. The SLI medical imaging system can image entire skin area of a person in multiple images or selected skin areas of interest. For example, a person's whole back area or arm area is imaged during an annual visit or checkup. The SLI medical imaging system processes the 3D surface map, detects skin features, processes the feature data and provides a report of the skin features and any warning signs. Though the imaging is performed at the physician's office, the analysis can be performed by a computer system onsite or offsite.

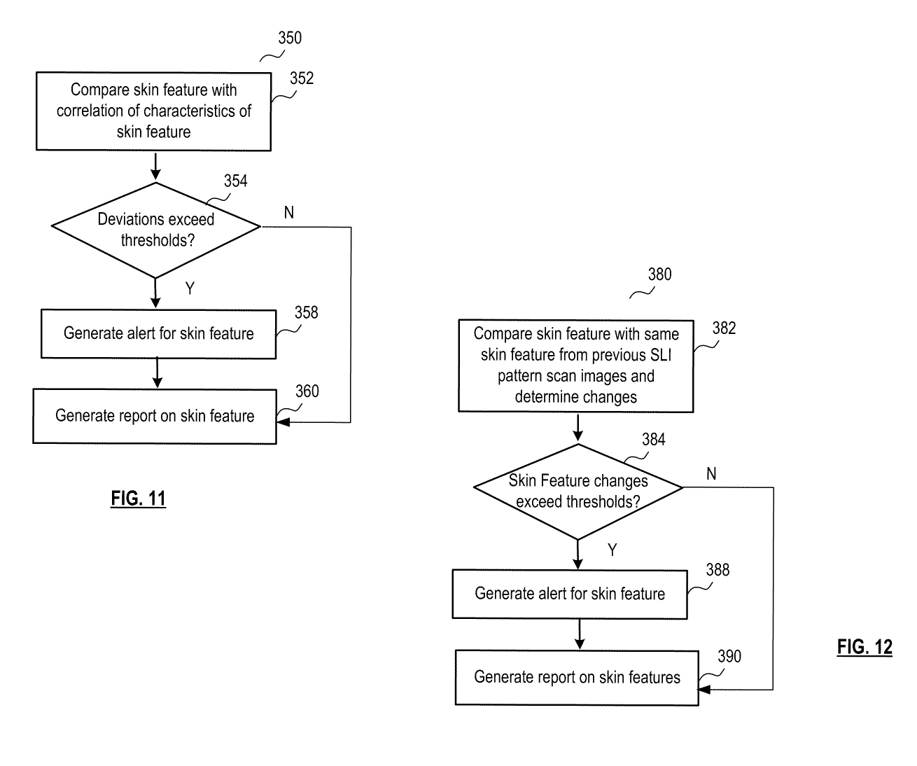

FIG. 11 illustrates a logic flow diagram of an embodiment of a method 350 for processing skin lesion data captured using SLI techniques. The SLI medical imaging system identifies a skin lesion as described herein. One or more characteristics of the feature data for a plurality of other skin lesions in the skin area is then correlated and each skin lesion compared to the correlation in step 352. For example, a correlation of color of skin lesions in a targeted skin area or a correlation of size and color of skin lesions in a targeted skin area is determined. In step 354, it is determined whether the feature data for a particular identified skin lesion includes deviations from the correlation that exceed a predetermined threshold. If so, an alert for the skin feature is generated in step 358. If not, a report on skin feature is generated in step 360 without the alert.

This process is sometimes called an “Ugly Duckling” analysis. The ugly duckling concept is the fact that skin lesions (in the same person) tend to be similar from one to another and those that are irregular may be malignant and should be checked. The analysis is very subjective when performed by a physician who views skin areas. The SLI medical imaging system provides a more objective process and analysis. A skin lesion that is flagged by the SLI medical imaging system can then be checked by a physician to determine further action.

FIG. 12 illustrates a logic flow diagram of an embodiment of another method 380 for processing skin feature data captured using SLI techniques. Another sign that a skin lesion is a melanoma is change in size, shape or color over time. The SLI medical imaging system provides objective measurements of skin lesions between scans. As described herein, a 3D surface map of a skin area is captured and 3D surface map is generated. Skin features are detected, such as moles, freckles, discolorations and other lesions, and feature data for selected lesions is determined, including location, size, shape, color of the skin feature. The feature data is then compared with feature data for the same skin feature from previous screenings in step 382. It is then determined in step 384, whether a skin feature has changed in size, shape or color exceeding an acceptable threshold. If so, an alert or flag of the skin feature is generated in step 388. The change in feature data is provided in a report by the SLI medical imaging system in step 390.

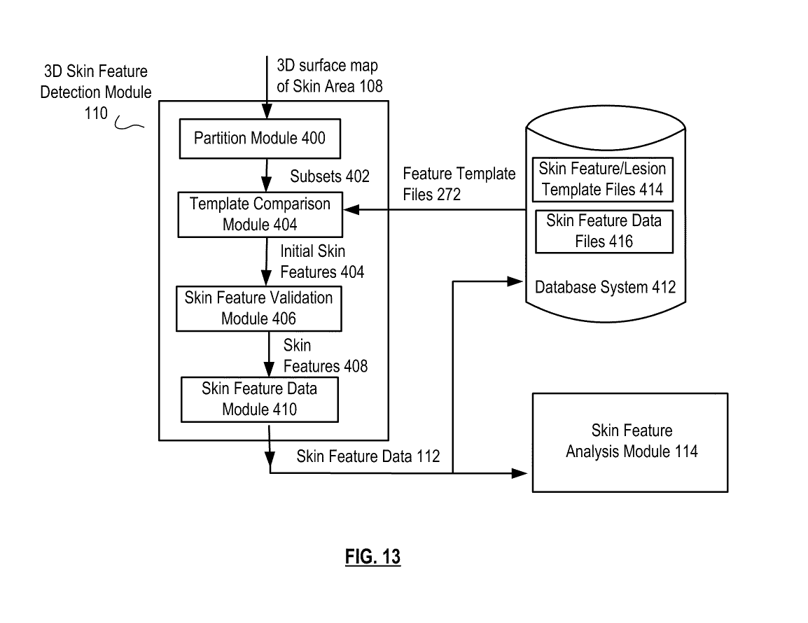

FIG. 13 illustrates a schematic block diagram of an embodiment of a skin feature detection module 110. In general, the skin feature detection module 110 includes one or more processing devices, such as a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. The skin feature detection module 110 includes a memory that is an internal memory or an external memory. The memory of the skin feature detection module 110 may each be a single memory device or a plurality of memory devices. Such a memory device may be a read-only memory, random access memory, volatile memory, non-volatile memory, static memory, dynamic memory, flash memory, cache memory, and/or any device that stores digital information. The skin feature detection module 110 may implement one or more of its functions via a state machine, analog circuitry, digital circuitry, and/or logic circuitry, the memory storing the corresponding operational instructions may be embedded within, or external to, the circuitry comprising the state machine, analog circuitry, digital circuitry, and/or logic circuitry. The skin feature detection module 110 may execute hard coded and/or software and/or operational instructions stored by the internal memory and/or external memory to perform the steps and/or functions illustrated in FIGS. 1 through 15 described herein.

The skin feature detection module 110 includes a partition module 400, template comparison module 404, skin feature validation module 406 and skin feature data module 410. Though the modules are shown as separate modules, one or more of the functions of the modules may be combined into another module or functions further segmented into additional modules. The skin feature detection module 110 and partition module 400, template comparison module 404, skin feature validation module 406 and skin feature data module 410 may be integrated into one or more devices or may be separate devices. The skin feature detection module 110 is coupled to a database system 412. The database system 412 stores skin feature template files 414 and skin feature data files 416.

In operation, the partition module 400 receives the 3D surface map 108 of the skin area. The 3D surface map 108 includes a plurality of points each having 3D coordinates. The 3D coordinates include for example Cartesian (x, y, z) coordinates, spherical (r, θ, Φ) coordinates or cylindrical (y, r, θ) coordinates. The 3D coordinates are in reference to an axis point defined in the surface map or other defined reference plane. Each of the points in the surface map 108 also includes texture data. For example, texture data includes color information such as RGB values or a brightness value or a grey level. The partition module 400 divides the 3D surface map 108 into subwindows or subsets 402 of the plurality of points. The subsets of points 402 may be exclusive or overlapping. This step is performed to ease processing of skin feature detection and may be eliminated depending on the application.

The template comparison module 404 processes the subsets of points 402 to detect one or more predetermined types of skin features. For example, skin lesions can be grouped into two categories: primary and secondary. Primary skin lesions are variations in color or texture that occur at birth, such as moles or birthmarks, or that may be acquired during a person's lifetime, such as those associated with infectious diseases (e.g. warts, acne, or psoriasis), allergic reactions (e.g. hives or contact dermatitis), or environmental agents (e.g. sunburn, pressure, or temperature extremes). Secondary skin lesions are those changes in the skin that result from primary skin lesions, either as a natural progression or as a result of a person manipulating (e.g. scratching or picking at) a primary lesion. The major types of primary lesions are:

Macule. A small, circular, flat spot less than ⅖ in (1 cm) in diameter. The color of a macule is not the same as that of nearby skin. Macules come in a variety of shapes and are usually brown, white, or red. Examples of macules include freckles and flat moles. A macule more than ⅖ in (1 cm) in diameter is called a patch.

Vesicle. A raised lesion less than ⅕ in (5 mm) across and filled with a clear fluid. Vesicles that are more than ⅕ in (5 mm) across are called bullae or blisters. These lesions may be the result of sunburns, insect bites, chemical irritation, or certain viral infections, such as herpes.

Pustule. A raised lesion filled with pus. A pustule is usually the result of an infection, such as acne, imptigeo, or boils.

Papule. A solid, raised lesion less than ⅖ in (1 cm) across. A patch of closely grouped papules more than ⅖ in (1 cm) across is called a plaque. Papules and plaques can be rough in texture and red, pink, or brown in color. Papules are associated with such conditions as warts, syphilis, psoriasis, seborrheic and actinic keratoses, lichen planus, and skin cancer.

Nodule. A solid lesion that has distinct edges and that is usually more deeply rooted than a papule. Doctors often describe a nodule as “palpable,” meaning that, when examined by touch, it can be felt as a hard mass distinct from the tissue surrounding it. A nodule more than 2 cm in diameter is called a tumor. Nodules are associated with, among other conditions, keratinous cysts, lipomas, fibromas, and some types of lymphomas.

Wheal. A skin elevation caused by swelling that can be itchy and usually disappears soon after erupting. Wheals are generally associated with an allergic reaction, such as to a drug or an insect bite.

Telangiectasia. Small, dilated blood vessels that appear close to the surface of the skin. Telangiectasia is often a symptom of such diseases as rosacea or scleroderma.

The major types of secondary skin lesions are:

Ulcer. Lesion that involves loss of the upper portion of the skin (epidermis) and part of the lower portion (dermis). Ulcers can result from acute conditions such as bacterial infection or trauma, or from more chronic conditions, such as scleroderma or disorders involving peripheral veins and arteries. An ulcer that appears as a deep crack that extends to the dermis is called a fissure.

Scale. A dry, horny build-up of dead skin cells that often flakes off the surface of the skin. Diseases that promote scale include fungal infections, psoriasis, and seborrheic dermatitis.

Crust. A dried collection of blood, serum, or pus. Also called a scab, a crust is often part of the normal healing process of many infectious lesions.

Erosion. Lesion that involves loss of the epidermis.

Excoriation. A hollow, crusted area caused by scratching or picking at a primary lesion.

Scar. Discolored, fibrous tissue that permanently replaces normal skin after destruction of the dermis. A very thick and raised scar is called a keloid.

Lichenification. Rough, thick epidermis with exaggerated skin lines. This is often a characteristic of scratch dermatitis and atopic dermatitis.

Atrophy. An area of skin that has become very thin and wrinkled. Normally seen in older individuals and people who are using very strong topical corticosteroid medication.

The template comparison module 404 detects one or more of these categories of skin lesions or other categories or types of skin lesions in the 3D surface map 108 or subset of points 402. Because the 3D surface map includes texture data, skin areas with color or grey levels that deviate from surrounding skin areas by a predetermined threshold are mapped. The 3D coordinates, size and shape of the detected skin area is also determined.

In an embodiment, the template comparison module 404 categorizes a detected skin lesion as a primary or secondary lesion and further categorizes the skin lesion into one or more of the described lesion types. The template comparison module 404 compares the detected skin lesions to one or more skin feature templates stored in the skin feature/lesion template files 414 and categorizes the detected skin lesions as one or more types of skin lesion.

In an embodiment, skin feature templates 414 are generated to correspond to one or more types of skin lesions described herein. To generate a skin feature template 414, a training dataset for the type of skin lesion is analyzed with a training algorithm to generate a feature vector or unique identifier for the type of skin lesion. The feature vector, such as an M×N vector, includes 3D coordinates and texture information. The training dataset includes a plurality of sets of 3D point clouds with texture data corresponding to the type of skin lesion. The training algorithm filters the dataset and creates a feature vector by reducing redundant information or removing extreme values. A training algorithm includes one or more of matched filters, correlation filters, Gabor filters (Gabor wavelets, log-Gabor wavelets) and Fourier transforms. A skin feature template includes a feature vector having one or more of: 3D coordinates for a skin lesion size, scale or shape, color and deviations and other feature data. In addition, for each feature, templates can be generated to further define sub-features.

The template comparison module 404 compares a subset of the 3D surface map with a feature vector. Again, matched filters, correlation filters, Gabor filters (with Gabor wavelets, log-Fabor wavelets) and Fourier transforms can be used to perform the comparison between the feature vector and detected skin lesion. Based on the comparison, the template comparison module generates a quality assessment value. In another embodiment, a multi-layered neural network can be implemented to process the skin lesion and determine a type of lesion.

The template comparison module 404 performs a subset by subset analysis for skin lesion detection and categorization. In another embodiment, subsets are selected for skin lesion detection based on a flow direction of color change or shape change in a skin area. Color or shape change direction measured with vectors fields are used to select the subsets for skin lesion detection. After a comparison with a feature template 414, a quality assessment value is assigned based on a probability or correlation that a skin lesion matches the lesion type. The template comparison module 404 generates the initial skin features data 404 that includes the quality assessment value and categorization.

The skin feature validation module 406 analyzes the quality assessment values assigned to skin lesions and determines a quality assessment. The skin feature validation module 406 adds another level of robustness to the overall system. The skin feature validation module 406 detects distinctions between lesion types in the 3D surface map. For example, when a quality assessment value falls below a threshold, the feature validation module 406 employs additional processing to determine whether the type of skin lesion is present in the location. In another embodiment, the feature validation module 406 further defines a type of skin lesion detected by the template comparison module. The skin feature validation module 406, for example, employs larger M×N feature vectors with additional information for a type of skin lesion and additional training vectors to further define and validate a type of skin lesion. The feature validation module 406 processes the skin lesions using one or more of the following methods: Principal Component Analysis (PCA), Independent component analysis (ICA), Linear discriminant analysis (LDA), Kernel-PCA, Support Vector Machine (SVM) or a Neural Network. For example, the feature validation module 406 processes a skin lesion detected by the template comparison module 404 and generates a PCA vector. The generated PCA vector is then compared with one or more feature vectors. A quality assessment is generated based on the comparison. The skin feature validation module 406 then generates the identified skin features 408 that have been detected and categorized.

The skin feature data module 410 analyzes the identified skin features 408 and generates feature data 112 for the identified skin features. The feature data 112 includes a list of skin lesions with 3D coordinates of points comprising the skin lesion as well as size, shape, color data and type of skin lesion. In an embodiment, the feature data 112 further includes relative placement of the skin lesion with respect to other skin lesions. For example, it may include a distance and an orientation angle of a skin lesion with respect to other skin lesions. This information assists in locating the skin lesion in future scans. The feature data 112 further includes a 3D scan image of the skin area and individual images or 3D surface maps of each detected skin lesion as well as 2D images. The 3D scan images allow a physician to later view the skin lesions in the skin area. The feature data for the skin area is stored in a feature data file in the database system.

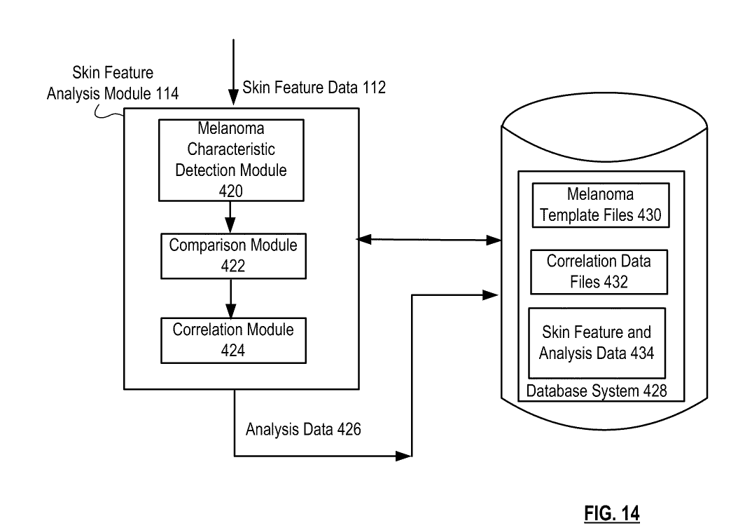

FIG. 14 illustrates a schematic block diagram of an embodiment of a skin feature analysis module 114. In general, the skin feature analysis module 114 includes one or more processing devices, such as a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. The skin feature analysis module 114 includes a memory that is an internal memory or an external memory. The memory of the skin feature analysis module 114 may each be a single memory device or a plurality of memory devices. Such a memory device may be a read-only memory, random access memory, volatile memory, non-volatile memory, static memory, dynamic memory, flash memory, cache memory, and/or any device that stores digital information. The skin feature analysis module 114 may implement one or more of its functions via a state machine, analog circuitry, digital circuitry, and/or logic circuitry, the memory storing the corresponding operational instructions may be embedded within, or external to, the circuitry comprising the state machine, analog circuitry, digital circuitry, and/or logic circuitry. The skin feature analysis module 114 may execute hard coded and/or software and/or operational instructions stored by the internal memory and/or external memory to perform the steps and/or functions illustrated in FIGS. 1 through 15 described herein.

The skin feature analysis module 114 includes a melanoma characteristic detection module 420, comparison module 422 and correlation module 424. Though the modules are shown as separate modules, one or more of the functions of the modules may be combined into another module or functions further segmented into additional modules. The skin feature analysis module 114 and melanoma characteristic detection module 420, comparison module 422 and correlation module 424 may be integrated into one or more devices or may be separate devices. The skin feature analysis module 114 is coupled to a database system 428. The database system 428 stores melanoma template files 430, correlation data files 432 and skin feature and analysis data files 434.

The skin feature analysis module 114 receives the skin feature data 112 for a 3D Surface Map of a skin area from the skin feature detection module 110. For each skin lesion identified in the skin feature data file, the melanoma characteristic detection module 420 processes the feature data for the skin lesion to determine whether the skin lesion includes one or more characteristics of melanoma. For example, known characteristics of a melanoma are sometimes referred to as ABCD characteristics. These characteristics include asymmetrical shape, irregular border, multiple colors and greater than 6 mm diameter. The skin feature data 112 for each detected skin lesion is processed to determine whether one or more of these characteristics is exhibited by the skin lesion. The skin feature analysis module 114 may use melanoma template files 430 stored in the database system 428. The melanoma template file includes a melanoma feature vector or unique identifier for a characteristic of a melanoma. The melanoma feature vector, such as an M×N vector, includes 3D coordinates and texture information can be compared and analyzed against the feature data for a skin lesion. In addition, since the skin feature data 112 includes 3D coordinates for each pixel in the 3D surface map of the skin lesion, the diameter, shape and border can be measured. Color changes exceeding a predetermined threshold within the area of the skin lesion can also be measured. Additional or alternative characteristics can also be measured using the 3D surface map of the skin lesion. The melanoma characteristic detection module 420 then generates any melanoma characteristic data for the identified skin features in the skin feature data 112.

The comparison module 422 compares a skin lesion identified in the skin feature data 112 with prior scans of the skin lesion. The skin lesion is detected in prior scans of a skin area by location and relative placement with respect to other skin lesions. The 3D coordinates and texture data from prior and current scans are compared and changes in size, shape and color of the skin lesion are measured. The changes in a skin lesion can thus be objectively measured over time.

The correlation module 424 processes feature data for skin lesions in a skin area and generates a correlation vector or feature template and stores the correlation data in the correlation data files 432. A selected skin lesion is then compared to the correlation to determine irregularities or abnormalities exceeding a threshold. For example, a skin lesion with color, size or shape that exceeds thresholds is flagged. This process is similar to the “Ugly Duckling” test performed by physicians.

The analysis data 426 from the melanoma characteristic detection module 420, comparison module 422 and correlation module 424 for the skin area is generated and stored in the database along with the skin feature data.

The SLI medical imaging system is applicable to other areas in the field of dermatology besides screening for melanoma. For example, the SLI medical imaging sensor can capture and process images to detect and monitor eczema, acne, wrinkles, blisters, discoloration and other skin conditions. Often, the effectiveness of a skin treatment is judged with only subjective data, such as viewing photographs of the affected skin area. The SLI medical imaging system provides an affordable tool to monitor changes in skin conditions over time.

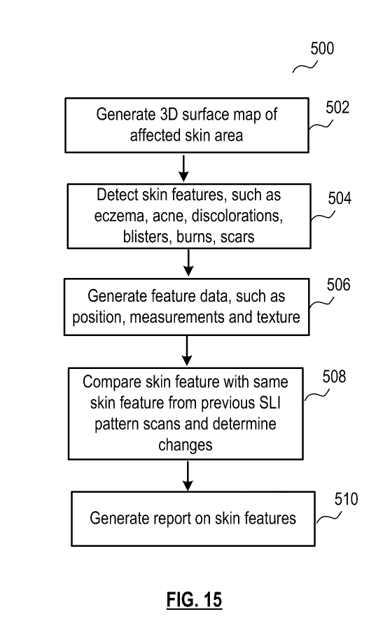

Though melanoma has been used as an example, similar processes as described herein may be used to detect and analyze other types of skin features such as eczema, acne, discolorations, blisters, burns and scars. For example, FIG. 15 illustrates a logic flow diagram for a method 500 for SLI imaging and monitoring of other types of skin lesions, such as eczema, acne, blisters, pigmentation and discolorations.

An SLI pattern image of an affected skin area is captured and processed to generate a 3D surface map of the skin area in step 502. The skin lesions, such as eczema, acne, discolorations, blisters, burns and scars, can be seen and extracted from the 3D surface map in step 504. Depending on the type of skin lesion, various feature data can be determined from the 3D surface map in step 506. For example, color, variations in color, pattern, shape, size and density can be measured. The measurements are then compared with prior measurements of the skin area in step 508. For example, in an embodiment, by comparing measurements over various periods of time, the effectiveness of a treatment can be determined with objective data. A report on changes over time from the comparison is generated in step 510.

In another embodiment, the SLI medical imaging system described herein will use one or more different wavelengths of light to project an SLI pattern at a subsurface of a skin lesion. The one or more wavelengths of light are able to penetrate a surface of a skin lesion and may be selected from infrared, visible, ultraviolet, x-ray or gamma ray spectrum of wavelengths of light. A camera sensitive to the one or more wavelengths of light will capture an image of the SLI pattern distorted by subsurface features of the skin lesion. A 3D surface map is generated from the images by analyzing the distortions in the SLI pattern. The 3D surface map will thus include subsurface features of the skin lesion. Subsurface features of a skin lesion, such as layers of growth of a mole subsurface, can then be analyzed.

Due to high costs, current imaging systems are not affordable for the average doctor's office. In addition, current imaging costs are too expensive for annual visits or regular check-ups. Due to its lower costs, the SLI medical imaging system described herein is affordable and cost effective solution for imaging at annual visits and check-ups. The SLI medical imaging system provides objective data about skin lesions, including growth and melanoma characteristics. The SLI medical imaging system can be used in addition to a physician's visual examination of skin areas.

As may be used herein, the term “operable to” indicates that an item includes one or more of processing modules, data, input(s), output(s), etc., to perform one or more of the described or necessary corresponding functions and may further include inferred coupling to one or more other items to perform the described or necessary corresponding functions.

The present invention has also been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claimed invention.

The present invention has been described above with the aid of functional building blocks illustrating the performance of certain significant functions. The boundaries of these functional building blocks have been arbitrarily defined for convenience of description. Alternate boundaries could be defined as long as the certain significant functions are appropriately performed. Similarly, flow diagram blocks may also have been arbitrarily defined herein to illustrate certain significant functionality. To the extent used, the flow diagram block boundaries and sequence could have been defined otherwise and still perform the certain significant functionality. Such alternate definitions of both functional building blocks and flow diagram blocks and sequences are thus within the scope and spirit of the claimed invention. One of average skill in the art will also recognize that the functional building blocks, and other illustrative blocks, modules and components herein, can be implemented as illustrated or by one or multiple discrete components, networks, systems, databases or processing modules executing appropriate software and the like or any combination thereof.

What is claimed is:

1. A structured light illumination (SLI) medical imaging system, comprising:
an SLI image sensor system that captures one or more two dimensional (2D) images of a skin area while a structured light pattern is projected onto the skin area;
a medical image processing module that receives the one or more 2D images and generates a three dimensional (3D) surface map of the skin area;
a feature detection module that identifies and categorizes a skin lesion from the 3D surface map of the skin area and generates feature data of the identified skin lesion; and
a feature analysis module that analyzes the feature data of the identified skin lesion to generate analysis data.

2. The SLI medical imaging system of claim 1, wherein the feature detection module generates feature data that includes texture data and position and size measurements of the identified skin lesion.

3. The SLI medical imaging system of claim 2, wherein the feature analysis module is operable to:
determine a correlation of one or more characteristics of a plurality of other identified skin lesions in the skin area of the 3D surface map;
compare the feature data of the identified skin lesion with the correlation of one or more characteristics of the other identified skin lesions to generate deviations of the feature data from the correlation;
determine whether the deviations exceed a predetermine threshold; and
generate a flag for the identified skin lesion when the deviations of the correlation exceed the predetermined threshold.

4. The SLI medical imaging system of claim 2, wherein the feature analysis module is operable to:
receive previous feature data of the identified skin lesion generated from a prior 3D surface map;
compare the feature data of the identified skin lesion with the previous feature data of the skin lesion; and
determine whether changes in the feature data exceed a predetermined threshold.

5. The SLI medical imaging system of claim 1, wherein the feature detection module comprises:
a template comparison module that compares a set of points of the 3D surface map to a skin feature template to identify the skin lesion and assign an initial category of the skin lesion with a quality assessment value.

6. The SLI medical imaging system of claim 5, wherein the skin feature template includes a feature vector, wherein each point of the vector includes 3D coordinates and texture information, corresponding to a type of skin lesion.

7. The SLI medical imaging system of claim 6, wherein the feature detection module further comprises:
a skin feature validation module that receives the initial category of the skin lesion with a quality assessment value; and
processes the set of points of the 3D surface map with one or more additional feature vectors to identify and categorize the skin lesion.

8. The SLI medical imaging system of claim 7, wherein the feature detection module further comprises:
a skin feature data module that receives the set of points of the 3D surface map of the identified skin lesion and generates feature data for the identified skin lesion, wherein the feature data includes 3D coordinates of points comprising the skin lesion, size of the skin lesion, shape of the skin lesion, color information of the skin lesion and relative placement of the skin lesion.

9. The SLI medical imaging system of claim 1, wherein the SLI image sensor system comprises:
a projection system for projecting the structured light pattern onto the skin area; and
a camera system for capturing the one or more 2D images of the skin area while the projection system projects the structured light pattern onto the skin area.

10. The SLI medical imaging system of claim 1, wherein the medical image processing module is operable to:
receive the one or more 2D images of the skin area;
segment pixels of object points from the one or more 2D images for processing; and
determine 3D coordinates and texture data from the segmented pixels of the object points to generate the 3D surface map of the skin area.

11. A method for processing images of a skin area by a processing module, comprising:
receiving a 3D surface map of a skin area for processing by a processing module;
identifying a skin lesion from the 3D surface map of the skin area and categorizing the identified skin lesion as one of a plurality of types of skin lesion by the processing module; and
generating feature data of the identified skin lesion from the 3D surface map of the identified skin lesion by the processing module, wherein the feature data includes texture data and position and size measurements of the identified skin lesion.

12. The method of claim 11, further comprising:
determining a correlation of one or more characteristics of a plurality of other identified skin lesions in the skin area of the 3D surface map;
comparing the feature data of the identified skin lesion with the correlation of one or more characteristics of the other identified skin lesions to generate deviations of the feature data from the correlation;
determining whether the deviations exceed a predetermine threshold; and
generating a flag for the identified skin lesion when the deviations of the correlation exceed the predetermined threshold.

13. The method of claim 12, further comprising:
receiving previous feature data of the identified skin lesion generated from a prior 3D surface map;
comparing the feature data of the identified skin lesion with the previous feature data of the skin lesion; and
determining whether changes in the feature data exceed a predetermined threshold.

14. The method of claim 11, wherein identifying a skin lesion from the 3D surface map of the skin area and categorizing the identified skin lesion as one of a plurality of types of skin lesion by the processing module, includes:
comparing a set of points of the 3D surface map to a skin feature template to identify the skin lesion and assign an initial category of the skin lesion with a quality assessment value, wherein the skin feature template includes a feature vector and wherein each point of the vector includes 3D coordinates and texture information, corresponding to a type of skin lesion.

15. The method of claim 11, further comprising:
receiving one or more two dimensional (2D) images of a skin area with a structured light pattern projected onto the skin area; and
generating the 3D surface map of the skin area from the 2D images.

16. The method of claim 15, further comprising:
segmenting pixels of object points from the one or more 2D images for processing; and
determining 3D coordinates and texture data from the segmented pixels of the object points to generate the 3D surface map of the skin area.

17. A method for imaging a skin area for screening for melanoma, comprising:
capturing one or more two dimensional (2D) images of a skin area with a structured light pattern projected onto the skin area;
generating the 3D surface map of the skin area from the 2D images, wherein each point of the 3D surface map includes 3D coordinates and texture data;
identifying a plurality of skin lesions from the 3D surface map of the skin area and categorizing the plurality of identified skin lesions as one of a plurality of types of skin lesion;
determining a correlation of one or more characteristics of the plurality of identified skin lesions in the skin area of the 3D surface map;
comparing one or more characteristics of one of the plurality of identified skin lesions with the correlation to generate deviations from the correlation;
determine whether the deviations exceed a predetermined threshold; and
generate a flag for the one of the plurality of identified skin lesions when the deviations of the correlation exceed the predetermined threshold.

18. The method of claim 17, further comprising:
determining feature data for the one of the plurality of identified skin lesions, wherein the feature data includes texture data and size measurements;
receiving previous feature data for the one of the plurality of identified skin lesions;
comparing the feature data for the one of the plurality of identified skin lesions with the previous feature data; and
determining whether changes in the feature data exceed a predetermined threshold.

19. The method of claim 18, further comprising:
processing the feature data for the one of the plurality of identified skin lesions to determine whether the one of the plurality of identified skin lesions includes one or more characteristics of melanoma, wherein the one or more characteristics of melanoma asymmetrical shape, irregular border, multiple colors and size approximately greater than 6 mm diameter.

20. The method of claim 19, further comprising:
providing analysis data for the one of the plurality of identified skin lesions, wherein the analysis data includes information on changes in the feature data exceeding a predetermined threshold, any detected characteristics of melanoma and whether the deviations exceed a predetermined threshold.
